(19) United States
(12) Patent Application Publication
(10) Pub. No.: US 2016/0296380 A1
(43) Pub. Date: Oct. 13, 2016

(54) TAMPON APPLICATOR

(71) Applicant: FIRST QUALITY HYGIENIC, INC., Great Neck, NY (US)

(72) Inventors: Christopher J. Graham, Lock Haven, PA (US); Nichlas Ward Brooks, Montoursville, PA (US); Daniel Wade Greenaway, Jersey Shore, PA (US); Eric J. Schall, Jersey Shore, PA (US)

(21) Appl. No.: 15/094,523

(22) Filed: Apr. 8, 2016

Related U.S. Application Data
(60) Provisional application No. 62/145,567, filed on Apr. 10, 2015.

(51) Int. Cl. A61F 13/26; A61F 13/20

(52) U.S. Cl. CPC A61F 13/266; A61F 13/202

(57) ABSTRACT

A tampon applicator assembly including a barrel and a plunger. The barrel includes an internal surface, an external surface, a proximal end portion and a distal end portion. A finger grip portion is disposed between the proximal end portion and the distal end portion. The internal surface includes at least one annular ridge within the finger grip portion and at least one longitudinally extending rib. The plunger is disposed within the barrel and includes flared proximal and distal end portions. The flared proximal end portion of the plunger has a larger circumference than a circumference of the at least one annular ridge of the barrel and the flared distal end of the plunger is in contact with the at least one longitudinally extending rib of the barrel.

### RELATED APPLICATION

This application is a non-provisional based on and claiming priority to U.S. Provisional Patent Application No. 62/145,567, filed Apr. 10, 2015, the contents of which are incorporated herein by reference in their entirety.

### FIELD OF THE INVENTION

The present disclosure relates to tampons, and in particular, to tampon applicators.

### BACKGROUND OF THE INVENTION

A tampon is a cylindrical mass of absorbent material used as a feminine hygiene product. Tampons are designed for insertion into the vagina during menstruation to absorb menstrual flow. One way of inserting tampons is to use a push-type applicator. These applicators generally include a pair of concentric, telescopically mounted hollow tubes: a barrel (the outer tube) and a plunger (the inner tube). The barrel contains the tampon and has a smooth surface to aid insertion. A user may push the plunger from its distal end to eject the tampon from the barrel into the vaginal canal. The tampon may be provided with an attached string, which extends outside of the barrel and through the plunger. Once the tampon has been inserted, the string extends outside the vagina, which can then be used to remove the tampon after use. Cordless tampons that are not visible when worn may also be used. Tampon applicators may be made of plastic, cardboard, or other comparable materials.

Conventional tampon applicators suffer from design flaws that have yet to be successfully addressed. For example, in current tampon applicators, the plunger is prone to accidental withdrawal from the barrel during normal use, which can be unsanitary and potentially dangerous. Further, in current tampon applicators, plungers can be pushed completely through the barrel and into the vagina during insertion, which can be a nuisance, unsanitary, or potentially dangerous to the user. Still further, current tampon applicators are prone to plunger misalignment that can damage the plunger, cause it to enter the barrel incorrectly, or in some cases, cause complete failure of the applicator assembly. Even if these design flaws are addressed, the result is often unsuccessful and/or associated with high manufacturing costs.

### SUMMARY OF THE INVENTION

Accordingly, an object of the present invention is to provide a tampon applicator that solves the problem of accidental plunger withdrawal, misalignment, accidental push through into the vagina, and failure, while simultaneously providing a simple design that is easier to manufacture as compared to conventional applicators.

The tampon applicator of the present invention includes two coaxially mounted hollow tubes: a plunger (inner tube) and barrel (outer tube). The barrel retains the tampon and the plunger is used to eject the tampon from the distal end of the barrel. The plunger may be a hollow tube that is tapered from its distal end to its proximal end. Both ends of the plunger may be flared, with the distal end having a larger diameter than the proximal end.

The barrel may be a hollow tube having a first opening at its proximal end and a second opening at its distal end. Located near the proximal end of the barrel is a finger grip. The finger grip is used to facilitate insertion of the barrel into the vagina. Inside the barrel's first opening are a plurality of ribs, which are raised bumps that protrude and terminate into the inner finger grip portion of the barrel. Located further into the barrel's inner finger grip portion is an annular ridge. The annular ridge may have a diameter smaller than that of the plunger's flared proximal end.

The ribs and the annular ridge are designed to interact in sliding relation with the plunger. The ribs serve to stabilize the plunger to prevent misalignment. The annular ridge, having a smaller diameter than the flared proximal end of the plunger, serves to prevent accidental plunger withdrawal from the barrel's distal end. The location of the annular ridge within the finger grip along with the ribs also limits the distance which the user can push the plunger, preventing the user from forcing the plunger back through the annular ridge.

When pushed inward into the barrel during use, the plunger's flared distal end ejects the tampon through the barrel's second opening, which may comprise a plurality of petals.

In some embodiments, the barrel has an internal surface, an external surface, a proximal end portion and a distal end portion. A finger grip portion is disposed between the proximal end portion and the distal end portion, where the internal surface comprises at least one annular ridge within the finger grip portion and at least one longitudinally extending rib.

In some embodiments, the at least one longitudinally extending rib extends from the proximal end portion to the finger grip portion.

In some embodiments, the at least one longitudinally extending rib comprises a plurality of longitudinally extending ribs.

In some embodiments, the plurality of longitudinally extending ribs are uniformly spaced from one another.

In some embodiments, the plurality of longitudinally extending ribs are non-uniformly spaced from one another.

In some embodiments, the finger grip portion comprises an hourglass shape.

In some embodiments, the finger grip portion comprises at least one rib on the external surface of the barrel.

In some embodiments, the at least one annular ridge comprises a plurality of annular ridges.

In some embodiments, the plurality of annular ridges are uniformly spaced from one another.

In some embodiments, the plurality of annular ridges are non-uniformly spaced from one another.

In some embodiments, the distal end portion comprises a plurality of petals that define an opening.

In some embodiments, a tampon applicator assembly has a barrel comprising an internal surface, an external surface, a proximal end portion and a distal end portion. A finger grip portion is disposed between the proximal end portion and the distal end portion, the internal surface comprising at least one annular ridge within the finger grip portion and at least one longitudinally extending rib. A plunger is disposed within the barrel, the plunger comprising flared proximal and distal end portions, the flared proximal end portion having a larger circumference than a circumference of the at least one annular ridge and the flared distal end being in contact with the at least one longitudinally extending rib.

In some embodiments, the tampon applicator assembly has at least one longitudinally extending rib extending from the proximal end portion to the finger grip portion.

In some embodiments, the at least one longitudinally extending rib comprises a plurality of longitudinally extending ribs.

In some embodiments, the plurality of longitudinally extending ribs are uniformly spaced from one another.

In some embodiments, the plurality of longitudinally extending ribs are non-uniformly spaced from one another.

In some embodiments, the finger grip portion comprises an hourglass shape.

In some embodiments, the finger grip portion comprises at least one rib on the external surface of the barrel.

In some embodiments, finger grip may have any suitable cross sectional shape, such as rectangular, triangular, ovoid, or spherical.

In some embodiments, the finger grip may have a plurality of uniformly or non-uniformly spaced circular ridges, grooves, pits, or the like, around the circumference of the barrel.

In some embodiments, the at least one annular ridge comprises a plurality of annular ridges.

In some embodiments, the plurality of annular ridges are uniformly spaced from one another.

In some embodiments, the plurality of annular ridges are non-uniformly spaced from one another.

In some embodiments, the distal end portion of the barrel comprises a plurality of petals that define an opening.

These and other features of this invention are described in, or are apparent from, the following detailed description of various exemplary embodiments of this invention.

### BRIEF DESCRIPTION OF THE DRAWINGS

The foregoing summary as well as the following detailed description of the exemplary embodiments of the present invention will best be understood when considered in conjunction with the accompanying drawings, wherein like designations denote like elements throughout the drawings and wherein:

FIG. 1 is a side view of the tampon applicator assembly according to an exemplary embodiment of the present invention.

FIG. 2 is a side view of the barrel according to an exemplary embodiment of the present invention.

FIG. 3 is a side view of the plunger according to an exemplary embodiment of the present invention.

FIG. 4 is a cross-sectional detail view of the barrel interior according to an exemplary embodiment of the present invention.

### DETAILED DESCRIPTION

The present invention relates to tampons, and in particular, to an improved tampon applicator assembly. The applicator assembly according to an exemplary embodiment of the present invention includes a barrel that has internal ribs. The internal ribs stabilize the plunger within the barrel and prevents the plunger from being accidentally withdrawn during use.

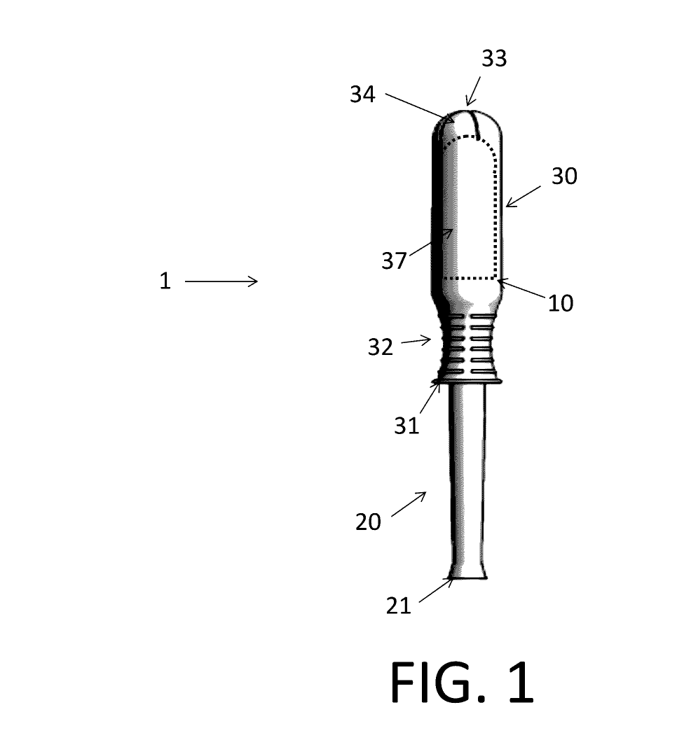

Referring to FIG. 1, a tampon applicator 1 is illustrated according to an exemplary embodiment of the present invention. Tampon applicator 1 includes a tampon 10, a plunger 20, and a barrel 30. Tampon 10 is retained inside barrel 30. Plunger 20 is used to eject tampon 10 from the distal end of barrel 30 into the vaginal canal.

Tampon 10 is preferably a cylindrical mass of absorbent material that may be made of materials such as, for example, cotton, rayon, or blends of rayon and cotton, to name a few. Rayon is made from wood pulp cellulose fibers. Surfactants or other finishes may also be used in tampon 10 as a process aid or to increase absorbency. Tampon 10 may be formed from a nonwoven process or contain components made from a nonwoven process. Nonwovens are widely used in the manufacture of tampons because they have outstanding mass per unit area and characteristics that are desirable in tampons, such as absorbency, resilience, softness, strength, and cushioning, to name a few. In some embodiments, tampon 10 may have a withdrawal string (not shown) that extends through plunger 20 and is long enough for a user to grasp after tampon 10 has been inserted into the vagina.

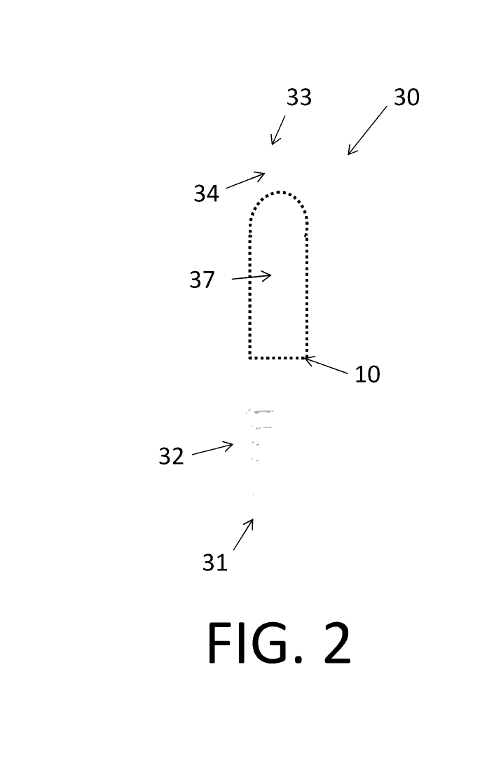

Referring now to FIG. 2, barrel 30 is shown. Barrel 30 retains tampon 10. Barrel 30 may be a hollow tube having a first opening 31 at its proximal end. Located near first opening 31 is a finger grip 32. Finger grip 32 is used to facilitate insertion of barrel 30 into the vagina. Preferably, a user may place finger grip 32 between a thumb and a middle finger for insertion, but any gripping method may be used. In an exemplary embodiment, finger grip 32 may have an hourglass shape. In yet other embodiments, finger grip 32 may have any suitable cross sectional shape, such as rectangular, triangular, ovoid, or spherical, to name a few. In other embodiments, finger grip 32 may have effectively the same diameter as the rest of barrel 30. In yet other embodiments, finger grip 32 may have a plurality of uniformly or non-uniformly spaced circular ridges, grooves, pits, or the like, around the circumference of the barrel to provide the user with enhanced grip. In other embodiments, a single ridge, groove, pit, or the like may be provided.

At the distal end of barrel 30 is a second opening 33, which includes a plurality of petals 34. The petals 34 are rounded and nearly converge at the top of barrel 30, leaving a small opening. Petals 34 are preferably designed such that minimal expulsion force is needed to expel tampon 10 through second opening 33 and petals 34, while still keeping tampon 10 in place such that it does not accidently eject through second opening 33 when stored. Barrel 30 may have any suitable diameter and may have a substantially cylindrical shape between finger grip 32 and petals 34. This portion of barrel 30 may be referred to as tampon retaining portion 37.

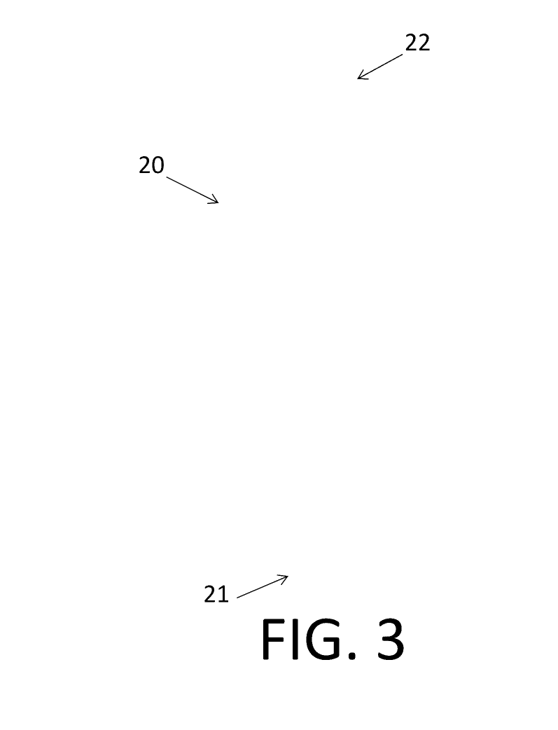

Referring now to FIG. 3, plunger 20 is shown. Plunger 20 is a hollow tube that is tapered along its length from its distal end to its proximal end. In other words, the distal end of plunger 20 has a larger diameter than its proximal end. At its proximal end, plunger 20 may have a finger flared portion 21. At its distal end, plunger 20 may have a tampon flare 22. In exemplary embodiments, finger flare 21 and tampon flare 22 are conical. In other embodiments, finger flare 21 and tampon flare 22 may have any suitable shape, such as rectangular, triangular, or spherical, or a combination thereof, to name a few.

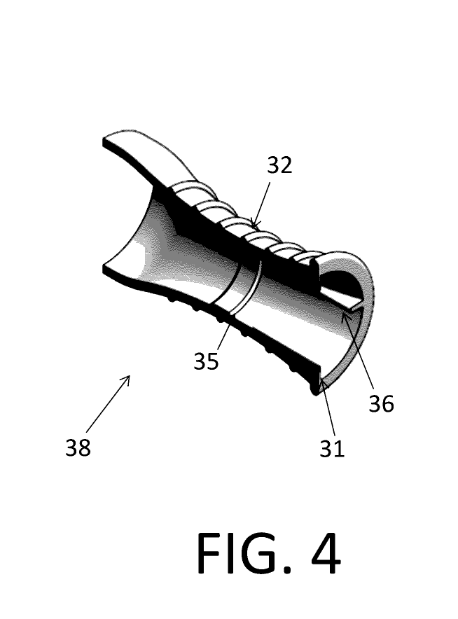

Referring now to FIG. 4, a detail view of the inner finger grip region 38 of barrel 30 is shown according to an exemplary embodiment of the present invention. Inner finger grip region 38 includes internal annular ridge 35 and longitudinally extending ribs 36. The combination of plunger 20, internal annular ridge 35, and ribs 36 produces a superior tampon applicator that solves the problems inherent in the prior art. Specifically, tampon applicator 1 solves the problem of accidental plunger withdrawal, plunger misalignment, and plunger failure, while providing a simpler design than the prior art that may be easier and cheaper to manufacture.

During the manufacture of tampon applicator 1, petals 34 are initially held open and finger flare 21 of plunger 20 is inserted first into barrel 30. Plunger 20 then moves downward through barrel 30 toward inner finger grip region 38.

Once finger flare 21 reaches inner finger grip region 38, finger flare 21 approaches internal annular ridge 35. In an exemplary embodiment, internal annular ridge 35 is circular and located at or near the narrowest interior portion of inner finger grip region 38. In other embodiments, internal annular ridge 35 may be a single ridge, broken ridge, dimple, or other suitable pattern. In yet other embodiments, internal annular ridge 35 may be a plurality of uniformly or non-uniformly spaced ridges, dimples, or other suitable patterns around the inner-circumference of the barrel. Finger flare 21 has a greater diameter than internal annular ridge 35. Thus, during manufacture, finger flare 21 is temporarily compressed as it passes through internal annular ridge 35. Once finger flare 21 has passed through internal annular ridge 35, finger flare 21 thereafter expands to its normal size and will prevent plunger 20 from accidental ejection through second opening 33 of barrel 30 during manufacturing and use.

At this point during the manufacturing process, plunger 20 has passed through first opening 31 and is now extended outside barrel 30. Tampon flare 22 prevents plunger 20 from accidental withdrawal through first opening 31 of barrel 30 because its diameter is considerably larger than the narrowest part of finger grip region 32 or internal annular ridge 35. Ribs 36 prevent plunger 20 from misalignment and serve to stabilize plunger 20 in barrel 30. Ribs 36 begin at first opening 31 and protrude into inner finger grip region 38. Ribs 36 may terminate at or before reaching internal annular ridge 35. In some embodiments, ribs 36 may be a plurality of dimples, ridges, or other suitable patterns. In other embodiments, a plurality of ribs 36 are uniformly or non-uniformly spaced around the inner-circumference of the barrel and first opening 31. In other embodiments, a single rib 36 may be used. In some embodiments, the channel formed by ribs 36 has a minimum diameter that is greater than the maximum diameter of tampon flare 22. Thus, ribs 36 do not compress tampon flare 22, but rather, serve only to assure proper alignment of plunger 20 in sliding relation with barrel 30. Ribs 36 also serve to prevent lateral wobble and stabilize plunger 20 such that its distal end, or widest portion, contacts ribs 36. Further, ribs 36 in conjunction with the location of internal annular ridge 35 prevents the user from forcing the plunger 20 beyond internal annular ridge 35 during use.

At this point in the manufacturing process, tampon 10 may be inserted into tampon retaining portion 37 as petals 34 are held open. Alternatively, tampon 10 may be inserted into barrel 30 simultaneously with plunger 20 as a sub-assembly. Once assembled, petals 34 are closed (which may include a post-forming step) and then tampon applicator 1 is ready for use. When a user pushes finger flare 21, tampon flare 22 contacts tampon 10 and ejects it through tampon retaining portion 37 and out of second opening 33. The tampon must overcome the minimum expulsion force of petals 34 as it exits second opening 33.

While particular embodiments of the invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A barrel for a tampon applicator, comprising:
an internal surface, an external surface, a proximal end portion and a distal end portion; and
a finger grip portion disposed between the proximal end portion and the distal end portion,
the internal surface comprising at least one annular ridge within the finger grip portion and at least one longitudinally extending rib.

2. The barrel of claim 1, wherein the at least one longitudinally extending rib extends from the proximal end portion to the finger grip portion.

3. The barrel of claim 1, wherein the at least one longitudinally extending rib comprises a plurality of longitudinally extending ribs.

4. The barrel of claim 3, wherein the plurality of longitudinally extending ribs are uniformly spaced from one another.

5. The barrel of claim 3, wherein the plurality of longitudinally extending ribs are non-uniformly spaced from one another.

6. The barrel of claim 1, wherein the finger grip portion comprises an hourglass shape.

7. The barrel of claim 1, wherein the finger grip portion comprises at least one rib on the external surface of the barrel.

8. The barrel of claim 1, wherein the at least one annular ridge comprises a plurality of annular ridges.

9. The barrel of claim 8, wherein the plurality of annular ridges are uniformly spaced from one another.

10. The barrel of claim 8, wherein the plurality of annular ridges are non-uniformly spaced from one another.

11. The barrel of claim 1, wherein the distal end portion comprises a plurality of petals that define an opening.

12. A tampon applicator assembly comprising:
a barrel comprising an internal surface, an external surface, a proximal end portion and a distal end portion;
a finger grip portion disposed between the proximal end portion and the distal end portion, the internal surface comprising at least one annular ridge within the finger grip portion and at least one longitudinally extending rib; and
a plunger disposed within the barrel, the plunger comprising flared proximal and distal end portions, the flared proximal end portion having a larger circumference than a circumference of the at least one annular ridge and the flared distal end being in contact with the at least one longitudinally extending rib.

13. The tampon applicator assembly of claim 12, wherein the at least one longitudinally extending rib extends from the proximal end portion to the finger grip portion.

14. The tampon applicator assembly of claim 12, wherein the at least one longitudinally extending rib comprises a plurality of longitudinally extending ribs.

15. The tampon applicator assembly of claim 14, wherein the plurality of longitudinally extending ribs are uniformly spaced from one another.

16. The tampon applicator assembly of claim 14, wherein the plurality of longitudinally extending ribs are non-uniformly spaced from one another.

17. The tampon applicator assembly of claim 12, wherein the finger grip portion comprises an hourglass shape.

18. The tampon applicator assembly of claim 12, wherein the finger grip portion comprises at least one rib on the external surface of the barrel.

19. The tampon applicator assembly of claim 12, wherein the at least one annular ridge comprises a plurality of annular ridges.

20. The tampon applicator assembly of claim 19, wherein the plurality of annular ridges are uniformly spaced from one another.

21. The tampon applicator assembly of claim 19, wherein the plurality of annular ridges are non-uniformly spaced from one another.

22. The tampon applicator assembly of claim 12, wherein the distal end portion of the barrel comprises a plurality of petals that define an opening.
